(19) United States
(12) Patent Application Publication
(10) Pub. No.: US 2025/0135177 A1
(43) Pub. Date: May 1, 2025

(54) SLIP LUER RETAINER

(71) Applicant: CareFusion 303, Inc., San Diego, CA (US)

(72) Inventor: Raymond P. FEITH, Chino Hills, CA (US)

(21) Appl. No.: 18/499,615

(22) Filed: Nov. 1, 2023

(51) Int. Cl. A61M 39/10

(52) U.S. Cl. CPC A61M 39/1011; A61M 2039/1083; A61M 2039/1088

(57) ABSTRACT

Luer retaining devices that provide increased retention between bearings and syringe tips are disclosed. A luer retaining device includes a male luer and a female luer. The male luer includes a syringe tip and the female luer includes a bearing that receives the syringe tip. The female luer includes at least one barb that extends from the bearing for engaging the syringe tip. The male luer and the female luer in fluid communication when the bearing receives the syringe tip.

### TECHNICAL FIELD

The present disclosure generally relates to slip luer retention in needleless access valves and, in some embodiments, to a slip luer retainer.

### BACKGROUND

Needle-free connectors, including neutral displacement needle-free connectors, offer a solution for providing medical fluid to patients. In an exemplary embodiment, a needle-free connector assembly promotes fluid transmission between a medical fluid supply and a catheter line. The medical fluid supply and the catheter line are secured to respective luers that are secured to a connector body. To transmit fluid, the luer connected to the medical supply is inserted into the connector body and overlaps a central post of the luer connected to the catheter line.

The overlapping between the luer and the central post can lead to issues. For example, movement of the luer relative to the central post, or vice versa, can cause contact between the luer and the central post, leading to increased risk of cracking or breaking the central post. When this occurs, the damaged area leads to fluid leakage, resulting in fluid loss and contamination.

Additionally, needleless access valves can inadvertently push a slip luer out. Disconnection of the slip luer and syringe tip may close the valve. This can occur when a compressed member disposed within the luer creates a force on the syringe tip. When such force is greater that the bearing friction, the syringe can be ejected.

### SUMMARY

In accordance with at least some embodiments disclosed herein is the realization that components used with slip luers and syringe tips can disconnect during use, which can result in a limited or closed valve and reduced medical delivery to a patient. When a slip luer and syringe tip disconnect, it may not be readily present to a medical professional that the component has separated, leading to delayed response times. Accordingly, there is a need for a needleless access valve with improved slip luer retention. This may be possible by including a locking hub to secure the slip luers and bearing.

Accordingly, aspects of the present disclosure provide a luer retaining device, comprising a male luer having a syringe tip, a female luer having a bearing for receiving the syringe tip, and at least one barb extending from the bearing for engaging the syringe tip, wherein the male luer and female luer are in fluid communication when the bearing receives the syringe tip. Some instances of the present disclosure provide an inner surface of the bearing. In some aspects, the barb extents around at least a portion of the inner surface. In some aspects, the barb extents around the entire inner surface.

In some aspects, the barb extends in a generally helical shape around the bearing. In some aspects, the syringe tip is rotatable relative to the bearing to control the engagement of the syringe tip and bearing. In some aspects, rotation of the syringe tip in a first direction tightens the engagement of the bearing and syringe tip. In some aspects, rotation of the syringe tip in the first direction locks the bearing relative to the syringe tip. In some aspects, rotation of the syringe tip in a second direction loosens the engagement of the bearing and syringe tip.

In some aspects, the syringe tip comprises threading on an outer surface. In some aspects, the threading extends in a generally helical shape around the syringe tip. In some aspects, the threading is configured to align with the barb. In some aspects, the barb comprises a first material and the syringe tip comprises a second material. In some aspects, the first material is harder than the second material. In some aspects, the bearing comprises a plurality of barbs. In some aspects, the plurality of barbs are evenly spaced apart on the bearing. In some aspects, the bearing comprises at least one row of barbs.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

The foregoing summary, as well as the following detailed description of embodiments of the device, will be better understood when read in conjunction with the appended drawings of an exemplary embodiment. It should be understood, however, that the invention is not limited to the precise arrangements and instrumentalities shown.

In the drawings:

FIG. 1 is a schematic view of an IV set coupled to a patient in accordance with an exemplary embodiment of the present invention;

FIG. 2 is a side cross section view of a fluid connector device in accordance with an exemplary embodiment of the present invention;

FIG. 3 is a top perspective cross section view of a second luer of the fluid connector device of FIG. 2;

FIG. 4 is a bottom perspective cross section view of a second luer of the fluid connector device of FIG. 2; and

FIG. 5 is a side cross section view of a second luer of the fluid connector device of FIG. 2.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an intravenous (“IV”) set, such embodiments can be used in other fluid conveyance systems. Furthermore, various applications of such embodiments and modifications thereto, which may occur to those who are skilled in the art, are also encompassed by the general concepts described herein.

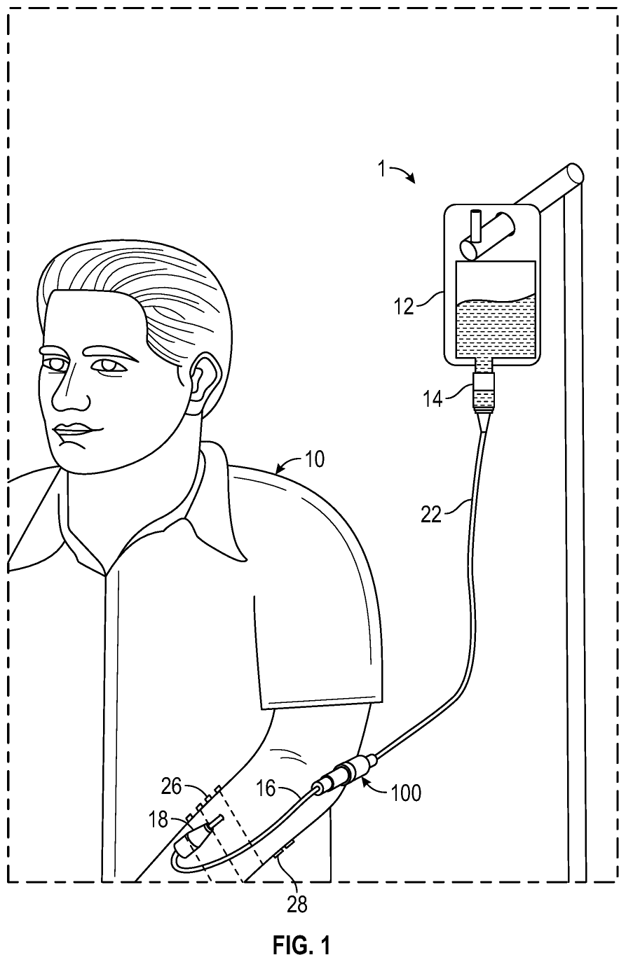

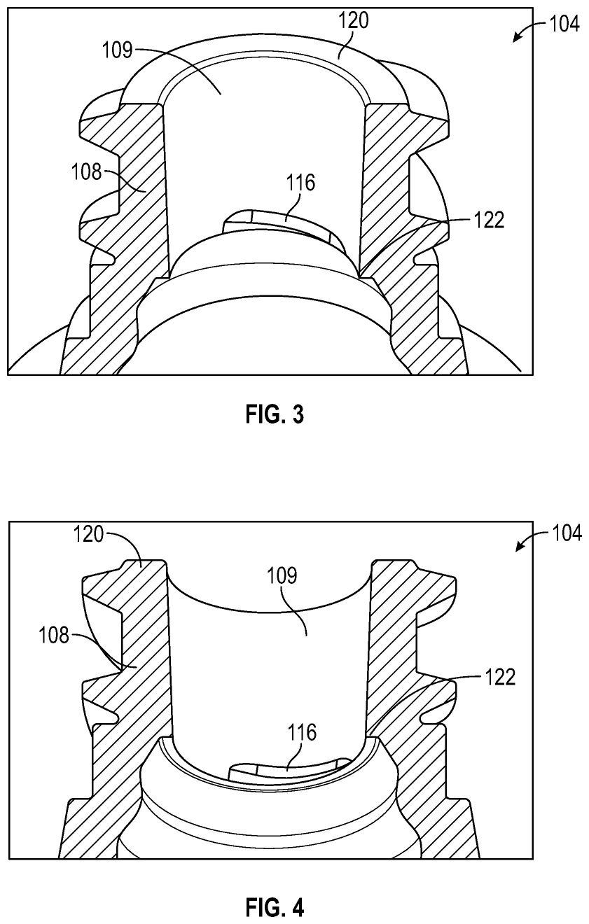

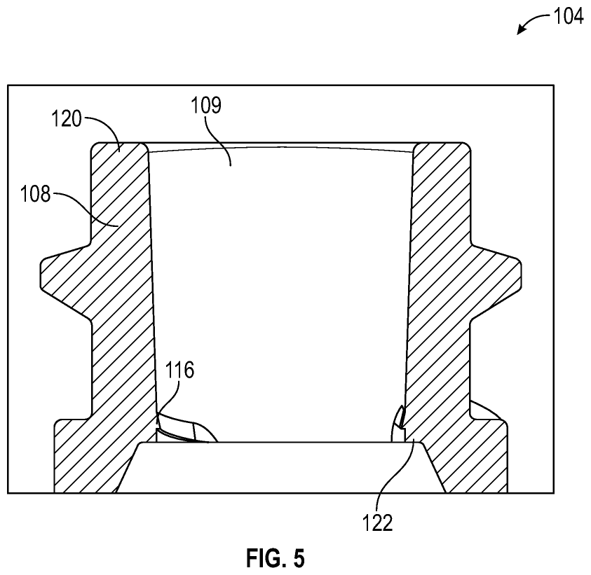

Referring to the drawings in detail, wherein like reference numerals indicate like elements throughout, there is shown in FIGS. 1-5 a device, generally designated 100, in accordance with an exemplary embodiment of the present invention.

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector device 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

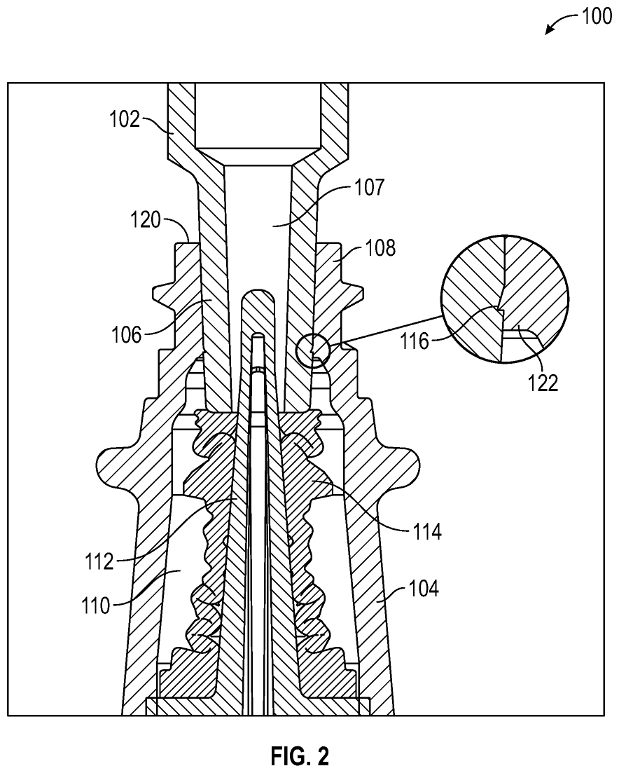

As shown in FIG. 2, the fluid connector device 100 may include a first luer 102 and a second luer 104. The first luer 102 and the second luer 104 may be in fluid communication when the first luer 102 is received in the second luer 104. The first luer 102 may be a male luer including a syringe tip 106. The first luer 102 may be referred to as a slip luer. The syringe tip may include a fluid channel 107 defined therein to allow a medical fluid to travel therethrough. The second luer 104 may be a female luer including a bearing 108, the bearing 108 shaped and sized to receive the syringe tip 106. The bearing 108 may be defined between a rim 120 and a ledge 122 of the second luer 104. The bearing 108 may be a generally cylindrical shape. In some embodiments, the bearing 108 may be conically angled to receive the syringe tip 106. The second luer 104 may include a cavity 110 and a post 112 defined therein. The post 112 may be received in the fluid channel 107 of the syringe tip 106 when the first luer 102 is received in the second luer 104.

The second luer 104 may include a compressible member 114 disposed around the post 112. The compressible member 114 may be movable between a compressed position and a decompressed position. The compressible member 114 may block a fluid inlet on the post 112 when the compressible member 114 is in the decompressed position. The compressible member 114 may prevent air, dust, medical fluid from entering the syringe tip when the compressible member 114 is in the decompressed position. The compressible member 114 may be moved into the compressed position when the first luer 102 is received in the second luer 104. The first luer 102 and the second luer 104 may be in fluid communication when the compressible member 114 is in the decompressed position. The compressible member 114 may generate a force opposing the first luer 102 when the compressible member 114 is in the decompressed position. If the force from the compressible member 114 acting on the first luer 102 is greater than a frictional force between the syringe tip 106 and the bearing 108, the first luer 102 may disconnect from the second luer 104.

Accordingly, it may be necessary to counteract the force acting on the first luer 102 to prevent the first luer 102 from separating from the second luer 104. As shown in FIGS. 2-6, the bearing 108 may include an inner surface 109 with at least one barb 116 extending therefrom. The barb 116 may extent from the inner surface 109 between 0.001-0.005 inches. In some embodiments, the barb 116 extends from the inner surface 109 between 0.0015-0.0045 inches, between 0.002-0.004 inches, or between 0.0025-0.0035 inches. The barb 116 may extent from the inner surface 109 at least 0.001 inch. In some embodiments, the barb 116 extends from the inner surface 109 at least 0.002, 0.003, 0.004 or 0.005 inches. The barb 116 may have a generally triangular cross section. The barb 116 may extend from the inner surface 109 proximate the ledge 122. In some embodiments, the barb 116 may extend from the inner surface 109 at a point between the rim 120 and the ledge 122. The barb 116 may have a first side and a second side. The first side may be closer to the ledge 122 on the inner surface 109 than the second side. The first side may extend a greater distance from the inner surface 109 the second side. In some embodiments, the second side may extend a greater distance from the inner surface 109 the first side. In some embodiments, the first side and the second side may extend approximately the same distance from the inner surface 109. In some embodiments, the barb 116 may have a generally rounded cross section.

The barb 116 may be molded onto the inner surface 109 during production of the second luer 104. The inner surface 109 may comprise a first material and the barb 116 may comprise a second material. The second material may be less deformable or harder than the first material. The barb 116 may have a generally helical configuration on the inner surface 109. In some embodiments, the barb 116 may be linear or non-helical. The barb 116 may be extent around at least a portion of the bearing 108. In some embodiments, the barb 116 extends around the entire bearing 108. The bearing 108 may include a plurality of barbs 116.

As shown in FIG. 5, the bearing 108 may include at least two barbs 116. The two barbs 116 may be evenly spaced relative to the other barb 116 on the inner surface 109. The two barbs 116 may be on a single row relative to the rim 120 and the ledge 122 on the inner surface 109. In some embodiments, the bearing 108 may include more than one row of barbs 116 (not shown) between the rim 120 and the ledge 122. In some embodiments the syringe tip 106 may include a receiving thread shaped and sized to receive the barb 116. In some embodiments, the syringe tip 106 includes a deformable outer surface that engages the barb 116. The barb 116 may engage an outer surface of the syringe tip 106. The outer surface of the syringe tip 106 may comprise a third material. The second material may be less deformable or harder than the third material. The helical configuration of the barb 116 on the inner surface 109 may allow for the syringe tip 106 to be tightened or loosened relative to the bearing 108 by rotating the syringe tip 106.

The syringe tip 106 may be rotated clockwise or counter-clockwise relative to the bearing 108 to tighten or loosen the engagement of the first luer 102 and the second luer 104. Rotation of the syringe tip 106 in a first direction relative to the bearing 108 may tighten the engagement of the first luer 102 and the second luer 104. Rotation of the syringe tip 106 in the first direction may lock the first luer 102 relative to the second luer 104. Rotation of the syringe tip 106 in a second direction relative to the bearing 108 may loosen the engagement of the first luer 102 and the second luer 104. The direction of rotation may be determined by the configuration of the barb 116 on the inner surface 109.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 18. The other clauses can be presented in a similar manner.
Clause 1: A luer retaining device, comprising a male luer having a syringe tip, a female luer having a bearing for receiving the syringe tip, and at least one barb extending from the bearing for engaging the syringe tip, wherein the male luer and female luer are in fluid communication when the bearing receives the syringe tip.
Clause 2: The luer retaining device of clause 1, further comprising an inner surface of the bearing.
Clause 3: The luer retaining device of clause 2, wherein the barb extents around at least a portion of the inner surface.
Clause 4: The luer retaining device of clause 2, wherein the barb extents around the inner surface of the bearing.
Clause 5: The luer retaining device of clause 1, wherein the barb extends in a generally helical shape around the bearing.
Clause 6: The luer retaining device of clause 5, wherein the syringe tip is rotatable relative to the bearing to control the engagement of the syringe tip and bearing.
Clause 7: The luer retaining device of clause 6, wherein rotation of the syringe tip in a first direction tightens the engagement of the bearing and syringe tip.
Clause 8: The luer retaining device of clause 7, wherein rotation of the syringe tip in the first direction locks the bearing relative to the syringe tip.
Clause 9: The luer retaining device of clause 6, wherein rotation of the syringe tip in a second direction loosens the engagement of the bearing and syringe tip.
Clause 10: The luer retaining device of clause 5, wherein the syringe tip comprises threading on an outer surface.
Clause 11: The luer retaining device of clause 10, wherein the threading extends in a generally helical shape around the syringe tip.
Clause 12: The luer retaining device of clause 11, wherein the threading is configured to align with the barb.
Clause 13: The luer retaining device of clause 1, wherein the barb comprises a first material and the syringe tip comprises a second material.
Clause 14: The luer retaining device of clause 13, wherein the first material is harder than the second material.
Clause 15: The luer retaining device of clause 1, wherein the bearing comprises a plurality of barbs.
Clause 16: The luer retaining device of clause 15, wherein the plurality of barbs are evenly spaced apart on the bearing.
Clause 17: The luer retaining device of clause 1, wherein the bearing comprises at least one row of barbs.
Clause 18: A luer retaining device, comprising a male luer having a syringe tip, a female luer having a bearing for receiving the syringe tip, and at least one barb extending from the bearing for engaging the syringe tip, wherein the male luer and female luer are in fluid communication when the bearing receives the syringe tip, wherein the barb extends in a generally helical shape around the bearing, and wherein the syringe tip is rotatable relative to the bearing to control the engagement of the syringe tip and bearing.
Clause 19: The luer retaining device of clause 18, wherein rotation of the syringe tip in a first direction tightens the engagement of the bearing and syringe tip.
Clause 20: The luer retaining device of clause 18, wherein rotation of the syringe tip in a second direction loosens the engagement of the bearing and syringe tip.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A luer retaining device, comprising:
a male luer having a syringe tip;
a female luer having a bearing for receiving the syringe tip; and
at least one barb extending from the bearing for engaging the syringe tip,
wherein the male luer and female luer are in fluid communication when the bearing receives the syringe tip.

2. The luer retaining device of claim 1, further comprising;
an inner surface of the bearing.

3. The luer retaining device of claim 2, wherein the barb extents around at least a portion of the inner surface.

4. The luer retaining device of claim 2, wherein the barb extents around the inner surface of the bearing.

5. The luer retaining device of claim 1, wherein the barb extends in a generally helical shape around the bearing.

6. The luer retaining device of claim 5, wherein the syringe tip is rotatable relative to the bearing to control the engagement of the syringe tip and bearing.

7. The luer retaining device of claim 6, wherein rotation of the syringe tip in a first direction tightens the engagement of the bearing and syringe tip.

8. The luer retaining device of claim 7, wherein rotation of the syringe tip in the first direction locks the bearing relative to the syringe tip.

9. The luer retaining device of claim 6, wherein rotation of the syringe tip in a second direction loosens the engagement of the bearing and syringe tip.

10. The luer retaining device of claim 5, wherein the syringe tip comprises threading on an outer surface.

11. The luer retaining device of claim 10, wherein the threading extends in a generally helical shape around the syringe tip.

12. The luer retaining device of claim 11, wherein the threading is configured to align with the barb.

13. The luer retaining device of claim 1, wherein the barb comprises a first material and the syringe tip comprises a second material.

14. The luer retaining device of claim 13, wherein the first material is harder than the second material.

15. The luer retaining device of claim 1, wherein the bearing comprises a plurality of barbs.

16. The luer retaining device of claim 15, wherein the plurality of barbs are evenly spaced apart on the bearing.

17. The luer retaining device of claim 1, wherein the bearing comprises at least one row of barbs.

18. A luer retaining device, comprising:
a male luer having a syringe tip;
a female luer having a bearing for receiving the syringe tip; and
at least one barb extending from the bearing for engaging the syringe tip,
wherein the male luer and female luer are in fluid communication when the bearing receives the syringe tip,
wherein the barb extends in a generally helical shape around the bearing, and
wherein the syringe tip is rotatable relative to the bearing to control the engagement of the syringe tip and bearing.

19. The luer retaining device of claim 18, wherein rotation of the syringe tip in a first direction tightens the engagement of the bearing and syringe tip.

20. The luer retaining device of claim 18, wherein rotation of the syringe tip in a second direction loosens the engagement of the bearing and syringe tip.
